(19) United States
(12) Patent Application Publication
(10) Pub. No.: US 2010/0273749 A1
(43) Pub. Date: Oct. 28, 2010

(54) Method for Treating Dry-Eye Syndrome

(76) Inventors: Stephen L. Sturley, New York, NY (US); Aaron Turkish, Bronx, NY (US); Stephen Trokel, New York, NY (US)

Correspondence Address: COOPER & DUNHAM, LLP, 30 Rockefeller Plaza, 20th Floor, NEW YORK, NY, 10112, US

(21) Appl. No.: 12/086,901

(22) PCT Filed: Dec. 28, 2006
(86) PCT No.: PCT/US2006/049570

§ 371 Date: Oct. 6, 2008

(51) Int. Cl. A61K 31/575; C07J 9/00; C07C 59/40; A61K 31/225; A61K 31/232; A61K 31/23; C07C 57/00; C07C 53/00; A61P 27/04

(52) U.S. Cl. 514/171; 552/541; 554/122; 514/182; 514/547; 514/549; 514/552; 554/121; 554/221; 554/1

(57) ABSTRACT

The subject application provides an aqueous composition suitable for use as an artificial tear solution comprising one or more lipids produced by an enzyme of the diacylglycerol acyltransferase 2 (DGAT2) family and/or the acyl-CoA cholesterol acytransferase (ACAT) family. This invention also provides related methods.

Throughout this application, various publications are referenced. Full citations for the publications relating to Examples 1-4 may be found immediately following Example 4. Full citations for the publications relating to Example may be found immediately following Example 5. The disclosures of these publications are hereby incorporated by reference into this application in order to more fully describe the state of the art as of the date of the invention described and claimed herein.

### BACKGROUND OF THE INVENTION

The production of tears is essential if the eyes are to function normally. There is a plethora of causes of impaired tear function ranging from mild to severe. Mild deficiencies of tears produce a subjective foreign body or gritty sensation, while more severe corneal deficiencies can lead to epithelial defects, corneal dryness and vision loss.

It has long been known that there are both aqueous and lipid components to the human tear film. Defects in both of these components are known to occur. The deficiency in tear production and quality is often age-related, and idiopathic, or of unknown cause. Sj\check{o}grens syndrome may be an identifiable cause as a primary condition or secondary and associated with a variety of immune system deficiencies.

Other known causes are local inflammations such as a dacryoadenitis, Grave's orbitopathy, or sarcoidosis.

Current treatment consists of attempting to identify the underlying cause, maximizing the efficacy of the tears that are present, and, in most cases, providing a substitute artificial tear to lubricate the eye. The use of topical 0.5% cyclosporine eye drops have proved efficacious in those patients in whom there is some degree of autoimmune suppression of tear production. Existing tears may have their efficacy maximized by the use of protective eyewear, tear duct plugs, and maintenance of a moist environment. Local palliative measures include treating any local blepharitis or allergic conjunctivitis.

However, in spite of these measures, to achieve ocular comfort and clear vision, artificial tears are usually required. The number of formulations of artificial tears is legion and reflects the attempts to mimic the human tear. A number of agents have been incorporated into the aqueous element to provide a simulation of the biphasic natural human tear. These include but have not been limited to hyaluronic acid, polyvinyl alcohol, mineral oil, polymethyl methacrylate, and a variety of vitamins and naturally occurring metabolites. However, all these formulations provide extremely short term relief.

Neutral lipids of a variety of forms represent the major lipid component of human tear film. To date, these lipids are known to be synthesized by the members of 3 independent gene families. All genes direct an esterification reaction between a free hydroxyl group (an alcohol such as sterol, diacylglycerol or long-chain waxy alcohols) and an acyl group (“fatty acid”) derived from a CoA-conjugate or a phospholipid). The Lecithin Cholesterol acyltransferase gene family conjugates sterols (and to a lesser extent diacylglycerols) with acyl groups derived from phosphatidylcholine and is primarily active in plasma.

Thus it likely plays a minimal role in tear film composition. By contrast the ACAT (Acyl-CoA Cholesterol acytransferase) and DGAT2 (diacylglycerol acyltransferease 2) gene family produce neutral lipids within the cell and are all expressed to varying extents in sebaceous glands (of which the Meibomian gland is a close derivative).

The human ACAT gene family has 3 members: ACAT1 synthesizes steryl esters in all tissues of the body, ACAT2 also produces steryl esters but is predominantly expressed in the liver and intestine, and DGAT1 produces triacylglycerol from diacylglyerol and fatty acylCoA, in most cell types (for comprehensive reviews of the primary literature see (Buhman at al., 2000; Farese, 1998; Sturley, 1997). The DGAT2 gene family is a set of seven genes that direct very similar reactions to that of the ACAT family, but bear no primary DNA sequence similarity. In general these enzymes do not esterify sterols, instead they take alcohols such as monoacylglycerol, (the monoacylglycerol acyltransferases, or MGATs), diacylglycerol (diacylglycerol acyltransferase 2 or DGAT2) long chain alcohols (the acylCoA wax alcohol acyltransferases, AWATs) and cojugate them to fatty acids derived from AcylCoenzyme A to make the corresponding neutral lipid (see table 1). Comprehensive reviews of the literature can be found at (Buhman et al., 2001; Oelkers and Sturley, 2004)

### SUMMARY OF THE INVENTION

The subject application provides an aqueous composition suitable for use as an artificial tear solution comprising one or more lipids produced by an enzyme of the diacylglycerol acyltransferase 2 (DGAT2) family and/or the acyl-CoA cholesterol acytransferase (ACAT) family.

The subject application also provides an aqueous composition suitable for use as an artificial tear solution comprising a lipid selected from the group consisting of a steryl ester, a triacylglycerol, a diacylglycerol, a wax ester and a dialcohol wax ester.

This application also provides a host-vector system comprising a host cell having therein (i) an expression vector encoding one or more enzymes of the DGAT2 family or (ii) a plurality of expression vectors, each vector encoding one enzyme of the DGAT2 family and/or ACAT family, wherein the host cell's genome does not encode the enzyme(s) encoded by the expression vector(s).

This application further provides a composition of matter comprising a plurality of host-vector systems, each host-vector system comprising a host cell having therein an expression vector encoding an enzyme of the DGAT2 family or the ACAT family, wherein the enzyme expressed in each host-vector system is different than that expressed in the other host-vector system(s), and wherein each host cell's genome does not encode the enzyme encoded by the expression vector therein.

This application also provides a method for producing an aqueous composition suitable for use as an artificial tear solution comprising admixing (i) an aqueous solution with (ii) one or more lipids produced by an enzyme of the DGAT2 and/or the ACAT families.

Finally, this application provides a method for producing an aqueous composition suitable for use as an artificial tear solution comprising admixing (i) an aqueous solution with (ii) a lipid selected from the group consisting of a triacylglycerol, a diacylglycerol, a waxy ester, a dialcohol wax ester, and a steryl ester.

### BRIEF DESCRIPTION OF THE FIGURES

FIG. 1: Evolutionary origin of the neutral lipid biosynthesis pathway. A: A dendogram of the ACAT gene family. B: A dendogram of the DGAT2 gene family. Phylogenetic trees indication the relatedness of ACAT and DGAT2 family sequences were constructed using the CLUSTLAW program (Macvector).

### DETAILED DESCRIPTION OF THE INVENTION

### Terms

As used herein, “administering” shall mean delivering in a manner which is effected or performed using any of the various methods and delivery systems known to those skilled in the art. Administering can be performed, for example, intravenously, orally, via implant, transmucosally, transdermally, intramuscularly, or subcutaneously. Specifically envisioned is topical administration to the eye via an eye drop dispenser. “Administering” can also be performed, for example, once, a plurality of times, and/or over one or more extended periods. For example, administration of the instant aqueous composition can comprise topical delivery of one, two or three drops of the composition via an eye drop dispenser, per eye, which delivery is performed once, twice, thrice or more per day, for one or more days as needed.

As used herein, “agent” shall include, without limitation, an organic compound, a nucleic acid, a polypeptide, a lipid, and a carbohydrate. Agents include, for example, agents which are known with respect to structure and/or function, and those which are not known with respect to structure or function.

As used herein, “AWAT1” shall mean acyl-CoA wax alcohol acyltransferase 1 and its synonym diacylglycerol acyltransferase 2 (DGA2). See, e.g. Sturley et al.

As used herein, “AWAT2” shall mean acyl-CoA wax alcohol acyltransferase 2 and its synonym human DGAT candidate gene 4 (hDC4).

As used herein, the members of the diacylgylcerol acyltransferase 2 (DGAT2) family are diacylglycerol acyltransferase 2 (DGAT2) (Genbank accession number AF384161), acyl-CoA monoacylglycerol acyltransferase 1 (MGAT1) (Genbank accession number AF157608), acyl-CoA monoacylglycerol acyltransferase 2 (MGAT2) (Genbank accession number AY157608), acyl-CoA monoacylglycerol acyltransferase 3 (MGAT3) (Genbank accession number AY229854), AWAT1 (Genbank accession number AY947638), AWAT2 (Genbank accession number AY605053), and human DGAT candidate gene 3 (hDC3) (Genbank accession number AL357752).

As used herein, the members of the Acyl-CoA Cholesterol acytransferase (ACAT) family are acylCoA cholesterol acyltransferase (ACAT1) (Genbank accession number BC028940), acylCoA cholesterol acyltransferase 2 (ACAT 2) (Genbank accession number AF059203), and diacylgylcerol acyltranferase 1 (DGAT1) (Genbank accession number AF059202).

As used herein, “pharmaceutically acceptable carriers” include, but are not limited to, 0.01-0.1 M and preferably 0.05 M phosphate buffer or 0.8% saline. Additionally, such pharmaceutically acceptable carriers can be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions and suspensions, including saline and buffered media. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like. Pharmaceutical carriers also include, for example, liposomes (see, e.g., U.S. Pat. No. 6,761,901 for examples of known liposomes).

As used herein, “subject” shall mean any animal, such as a primate, mouse, rat, guinea pig or rabbit. In the preferred embodiment, the subject is a human.

As used herein, “therapeutically effective amount” means an amount sufficient to treat a subject afflicted with a disorder or a complication associated with a disorder. Therapeutically effective amounts are readily determined based on animal data, and are exemplified by the various formulae herein below in conjunction with the embodiments for administration herein above.

As used herein, “treating” a subject afflicted with a disorder shall mean slowing, stopping or reversing the disorder's progression, and/or slowing, stopping or reversing the disorder's symptoms such as physical discomfort (e.g. irritation) or cosmetic abnormalities (e.g. red eye). In the preferred embodiment, treating a disorder means reversing the disorder's progression and/or reversing the progression of symptoms, ideally to the point of eliminating the disorder and/or symptoms.

As used herein, “transition temperature” means the temperature at which a lipid changes from the crystalline to the liquid state. It is a direct reflection of the chemical properties (saturation and acyl or alcohol chain length) of the lipids produced, which by inference represents the substrates provided to the reactions. A liquid state will be clear whereas the solid state will be, at worst, opaque.

### Embodiments of the Invention

The subject application provides an aqueous composition suitable for use as an artificial tear solution comprising one or more lipids produced by an enzyme of the diacylglycerol acyltransferase 2 (DGAT2) family and/or the acyl-CoA cholesterol acytransferase (ACAT) family.

In one embodiment, the composition comprises lipids produced by two enzymes of the DGAT2 and/or ACAT families. In another embodiment, the composition comprises lipids produced by three enzymes of the DGAT2 and/or ACAT families. In a further embodiment, the enzyme is acyl-CoA wax alcohol acyltransferase 1 (AWAT1) or acyl-CoA wax alcohol acyltransferase 2 (AWAT2). In an additional embodiment, the enzymes are AWAT1 and AWAT2. In a further embodiment, the composition further comprises (i) a preservative, (ii) a salt and/or (iii) a buffering agent. In an embodiment, the composition comprises (i) a preservative, (ii) a salt, and (iii) a buffering agent.

The subject application also provides an aqueous composition suitable for use as an artificial tear solution comprising a lipid selected from the group consisting of a steryl ester, a triacylglycerol, a diacylglycerol, a wax ester and a dialcohol wax ester.

In an embodiment, the steryl ester is approximately 77 percent saturated. In another embodiment, the triacylglycerol is approximately 38 percent saturated. In yet another embodiment, the waxy ester is approximately 23 percent saturated.

In a further embodiment, the composition further comprises (i) a preservative, (ii) a salt and/or (iii) a buffering agent. In an embodiment, the composition comprises (i) a preservative, (ii) a salt, and (iii) a buffering agent.

The subject application further provides an article of manufacture comprising an eye drop dispenser having therein any aqueous composition disclosed herein.

In an embodiment, the article of manufacture further comprises instructions for topically administering the aqueous composition to a subject's eye.

In another embodiment, the article of manufacture comprising an eye drop dispenser having therein any aqueous composition disclosed herein. In one embodiment, the article of manufacture further comprises instructions for topically administering the aqueous composition to a subject's eye.

This application also provides a method for lubricating the surface of a subject's eye comprising topically administering an effective amount of any composition of disclosed herein to the subject's eye.

In one embodiment of the method for lubricating the surface of a subject's eye, the subject is undergoing, or is about to undergo, an ocular procedure. In another embodiment of the method for lubricating the surface of a subject's eye, the subject is a human.

This application further provides a method for lubricating the surface of a subject's eye comprising topically administering an effective amount of any composition disclosed herein to the subject's eye. In one embodiment, the subject is undergoing, or is about to undergo, an ocular procedure. In another embodiment, the subject is a human.

This application further provides a method for treating a subject afflicted with dry eye syndrome comprising topically administering a therapeutically effective amount of any composition disclosed herein to one or both of the subject's eyes. In an embodiment, the subject is undergoing, or is about to undergo, an ocular procedure. In a further embodiment, the subject is a human.

This application also provides a host-vector system comprising a host cell having therein (i) an expression vector encoding one or more enzymes of the DGAT2 family or (ii) a plurality of expression vectors, each vector encoding one enzyme of the DGAT2 family and/or ACAT family, wherein the host cell's genome does not encode the enzyme(s) encoded by the expression vector(s).

In an embodiment, the host-vector system comprises a host cell having therein an expression vector encoding acyl-CoA wax alcohol acyltransferase 1 (AWAT1) and/or acyl-CoA wax alcohol acyltransferase 2 (AWAT2).

In another embodiment, the host-vector system comprises host cell having therein a first expression vector encoding AWAT1 and a second expression vector encoding AWAT2.

In another embodiment, the host cell is a yeast cell. In a further embodiment, the yeast cell is a quadruple mutant strain.

In a further embodiment, the host-vector system utilizes a bacterial, mammalian cell culture, or insect/recombinant baculovirus expression system.

This application further provides a composition of matter comprising a plurality of host-vector systems, each host-vector system comprising a host cell having therein an expression vector encoding an enzyme of the DGAT2 family or the ACAT family, wherein the enzyme expressed in each host-vector system is different than that expressed in the other host-vector system(s), and wherein each host cell's genome does not encode the enzyme encoded by the expression vector therein.

In one embodiment of the composition of matter, the composition comprises a first host-vector system wherein the expression vector encodes AWAT1, and a second host-vector system wherein the expression vector encodes AWAT2. In another embodiment of the composition of matter, each host cell is a yeast cell.

This application also provides a method for producing lipid comprising the steps of culturing any host-vector system disclosed herein in the presence of one or more lipid reactants and recovering from the lipid so produced any lipid whose production is catalyzed by DGAT2 family and/or an ACAT family enzyme.

This application further provides a method for producing lipid comprising the steps of culturing any composition disclosed herein in the presence of one or more lipid reactants, and recovering from the lipid so produced any lipid whose production is catalyzed by a DGAT2 family and/or an ACAT family enzyme.

This application also provides a method for producing an aqueous composition suitable for use as an artificial tear solution comprising admixing (i) an aqueous solution with (ii) one or more lipids produced by an enzyme of the DGAT2 and/or the ACAT families.

This application also provides a method for producing an aqueous composition suitable for use as an artificial tear solution comprising admixing (i) an aqueous solution with (ii) the lipid produced by any method disclosed herein.

This application further provides method for producing an aqueous composition suitable for use as an artificial tear solution comprising admixing (i) an aqueous solution with (ii) the lipid produced by any method disclosed herein.

Finally, this application provides a method for producing an aqueous composition suitable for use as an artificial tear solution comprising admixing (i) an aqueous solution with (ii) a lipid selected from the group consisting of a triacylglycerol, a diacylglycerol, a waxy ester, a dialcohol wax ester, and a steryl ester.

Dosages will be determined empirically based on the solubility and efficacy of the lipids being tested.

This invention is illustrated in the Examples section which follows. This section is set forth to aid in an understanding of the invention but is not intended to, and should not be construed to, limit in any way the invention as set forth in the claims which follow thereafter.

### EXAMPLES

### Example 1

### Selected Advantages of the Invention

Introduction

It is believed that the lipid component of natural human tears is an essential component of human tears and is required for a stable tear film. Human meibomian secretion is a waxy ester that liquefies between 30 and 35 degrees Celsius. The waxy ester forms a dam along the edge of the lower lid that helps hold the tear film in. The lipid coat reduces evaporation and maintains the essential lubrication of the epithelial surface. This prevents drying and the secondary manifestations of dryness.

It is predicted that this high melting point waxy ester is an essential component of natural or artificial tears. It is the premise of this invention that the products of the lipid genes discussed herein are critical to tear film function and that this can be recreated in vitro at a production scale level.

Advantages

The invention herein disclosed includes artificial formulations of tear film based on recombinant DNA approaches. These formulations are lipid-based and are intended to prevent dry-eye syndrome and other corneal disorders. These formulations can be used to treat any condition resulting in excessive corneal dehydration, including the application of contact lenses. Such excessive corneal dehydration can be age-related, idiopathic, or of unknown cause. Sj\check{o}grens syndrome may be an identifiable cause as a primary condition or secondary and associated with a variety of immune system deficiencies. Other known causes are local inflammations such as dacryoadenitis, Grave's orbitopaty, or sarcoidosis.

Human tear film is a critical factor in protection of the cornea from dessication and damage. A major molecular component of this body fluid is lipids which presumably are critical to the lubrication of this surface. These formulations can be mimicked and manipulated to produce an effective and useful topical application for a sufferer of disorders such as dry-eye syndrome, and also as a daily application for allergy sufferers or individuals using contact lenses. They will also be particularly useful as the eye repairs after major surgery. The enzymes that produce these lipids are expressed in a recombinant DNA expression system.

Here, model systems were developed to study the synthesis and homeostasis of several lipid classes, particularly the non-polar or neutral lipids. This latter category includes the fatty acyl ester derivatives of cholesterol and related sterols (Steryl esters, SE), monoacylglycerols (diglyceride (DG), diacylglycerols (triglycerides (TG), and long chain alcohols (wax esters)). These molecules are produced by a similar enzymatic reaction, that is, an acyltransferase reaction to create an ester bond between an alcohol (OH) and an acyl group of varying degrees of chain length and saturation (presence of double bonds). The human genes that determine the production of these acyltransferase enzymes have all been identified in the inventors' laboratory (See Table 1). It is noted that all non-human (e.g. mouse) genes for these enzymes are envisioned as embodiments. Furthermore, the expression of these enzymes has been performed in a recombinant form such that the individual lipid products can be produced, isolated and/or manipulated with regard to individual lipid composition. These lipids can be used as purified components of tear formulations or can be utilized as mixtures after combination with the other lipid classes described here. In addition, the opportunity exists to manipulate the chemical and physical properties of these formulations by controlling the chain length and/or degree of saturation of the lipid substrates (alcohol or fatty-acyl, or both) for these reactions. This will likely change the stability and solubility of these products in the formulation. Most importantly, this approach will provide the opportunity to control and manipulate the phase-transition temperature of these lipids.

TABLE 1
Human and yeast genes or Neutral lipid
biosynthesis. TG; triacylglycerol; DAG,
diacylglycerol; WE, wax esters (acylated
long chain alcohols); WDE, dialcohol
wax esters (acylated long chain di-alcohols);
SE, steryl esters; RE, retinyl esters. Gene
designations are as described in the body
of this application or in the scientific
literature (e.g. Buhman et al., 2000; Buhman
et al., 2001; Oelkers and Sturley, 2004).

| Gene | | | Gene | Lipid |
|---|---|---|---|---|
| (organism) | Chromosome | Genbank | family | product |
| ARE1 | III | | ACAT | SE |
| (yeast) | | | | |
| ARE2 | XIV | | ACAT | SE, TG |
| (yeast) | | | | |
| LRO1 | XIV | | LCAT | TG |
| (yeast) | | | | |
| DGA1 | XIII | | DGAT2 | TG |
| (yeast) | | | | |
| ACAT1 | 1q25 | BC028940 | ACAT | SE |
| (human) | | | | |
| ACAT2 | 12q13 | AF059203 | ACAT | SE |
| (human) | | | | |
| DGAT1 | 8q24.3 | AF059202 | ACAT | TG, RE |
| (human) | | | | |
| hDGAT2 | 11q13.5 | AF384161 | DGAT2 | TG |
| (human) | | | | |
| AWAT1 | Xq13.1 | AY947638 | DGAT2 | TG, WE |
| (human) | | | | |
| hDC3 | Xq13.1 | AL357752 | DGAT2 | TG, WE, WDE |
| (human) | | | | |
| AWAT2 | Xq13.1 | AY605053 | DGAT2 | TG, WE |
| (human) | | | | |
| MGAT1 | 2q36.3 | AF384163 | DGAT2 | DAG, TG |
| (human) | | | | |
| MGAT2 | 11q13.5 | AY157608 | DGAT2 | DAG, TG |
| (human) | | | | |
| MGAT3 | 7q22.1 | AY229854 | DGAT2 | DAG, TG |
| (human) | | | | |

A key component of this invention lies in the expression of these genes and their encoded enzymes and thus lipid products (Table 2) in the genetically malleable host, budding yeast or Saccharomyces cerevisiae as a recombinant production device. The organism has been genetically manipulated to lack the endogenous enzymes for the analogous neutral lipid biosynthetic reactions. These strains (and derivatives thereof, described in table 2), therefore present a unique resource, i.e. a host devoid of background enzymatic activity such that after introduction of the recombinant human enzymes the only neutral lipid produced will be that derived from the expressed human gene(s). This represents a major advantage in the production and purification of these lipids, while the yeast and human enzymes for these reactions are distinct, they will produce lipids with properties so similar that biochemical separation would likely be impossible or at least problematic.

Purification of the recombinant lipids will be achieved by conventional means. Recombinant yeast will be grown under conditions (media composition, temperature, aeration) and scales (mls to litres) that are optimized for yield of recombinant protein and or neutral lipid. In this organism, as in humans, the neutral lipid products of these enzymes are stored in cytoplasmic lipid droplets. Cells will be lysed using state of art procedures and due to their inherent buoyancy, the neutral lipid particles will be isolated by density centrifugation. Depending on requirements, the lipids can be further purified following proteolysis and/or organic solvent extraction, followed by reconstitution into the appropriate carrier. The precise formulation will require empirical determination.

Similarly, human Meibomian gland secretions (the tear film) are of heterogeneous composition, in terms of varied lipids and proteins, the necessity and function of which are objects of speculation. However, the experimental potential exists to combine these in any manner in a controlled fashion.

The genes will be expressed in the aforementioned hosts. Multiple genes can be coexpressed. Lipids will be purified and combined as deemed appropriate. The efficacy of these preparations will be tested in appropriate murine models of dry-eye syndrome. (See e.g. Barabino S, Shen L, Chen L, Rashid S, Rolando M, Dana M R, Invest Opthalmol Vis Sci. 2005 August, 46(8):2766-71; Karan G, Lillo C, Yang Z, Cameron D J, Locke K G, Zhao Y, Thirumalaichary S, Li C, Birch D G, Vollmer-Snarr H R, Williams D S, Zhang K, Proc Natl Acad Sci USA. 2005 Mar. 15, 102(11):4164-9; and Barabino S, Dana M R. Animal models of dry eye: a critical assessment of opportunities and limitations, Invest Opthalmol V is Sci. 2004 June, 45(6):1641-6.)

TABLE 2
Lipid composition of meibomian gland secretions and candidate
genes. TG; triacylglycerol; DAG, diacylglycerol; WE, wax esters
(acylated long chain alcohols); WDE, dialcohol wax esters
(acylated long chain di-alcohols); SE, steryl esters.

| | | | | | |
|---|---|---|---|---|---|
| | | | | | Yeast |
| | Mean % | Fatty acid | Acyl Group | | Expression |
| Lipid | Meibomian | Composition | Chain | Responsible | Vector |
| Class | fluid | (double bond) | length | Gene | Reference |

| | | | | | |
|---|---|---|---|---|---|
| SE | 27 (8-34) | 77 % Saturated | 17:0 (10%) | ACAT1 | Oelkers |
| | | | 20:0 (6%) | or | et al., |
| | | | 24:0 (12%) | ACAT2 | |
| | | | 26:0 (14%) | | |
| SE | | 23% Unsaturated | 18:1 (3%) | | 1998; |
| | | | 22:1 (2%) | | Yang et |
| | | | 24:1 (7%) | | al., 1997 |
| | | | 26:1 (2%) | | |
| TG | 3.7 (11-43) | 38% Saturated | 16:0 (15%) | DGAT1 | Oelkers |
| | | | 17:0 (3%) | DGAT2 | et al., 1998; |
| | | | 18:0 (4%) | AWAT1 | Yang et |
| | | | | | al., 1997 |
| | | 62 % Unsaturated | 16:1 (9%) | AWAT2 | Turkish |
| | | | 18:1 (44%) | hDC3 | et al., |
| | | | | MGAT1, 2, 3 | 2004 |
| DAG | 3.0 | NA | NA | MGAT1, 2, 3 | |
| WE | 32 (13-23) | 23% Saturated | 16:0 (6%) | AWAT1 | Turkish |
| | | | 17:0 (10%) | | et al., |
| | | | 18:0 (12%) | | 2004 |
| | | 77% Unsaturated | 16:1 (10%) | AWAT2 | |
| | | | 18:1 (56%) | | |
| WDE | 8 | NA | NA | hDC3 | |

### Example 2

### Exemplary Lipid Combinations

Table 3 is a list of selected embodiments of the instant composition. Specifically, each aqueous composition in Table 3 is identified by a number from 1-118. Each such composition comprises one or more lipids as indicated by the boxes marked with an “X.” The indicators “DGAT2”, “MGAT1”, “MGAT2”, MGAT3″, “AWAT1”, “AWAT2”, and “hDC3” represent, in this Table, lipids produced by the enzymes diacylglycerol acyltransferase 2, acyl-CoA monoacylglycerol acyltransferase 1, monoacylglycerol acyltransferase 2, monoacylglycerol acyltransferase 3, acyl-CoA wax alcohol acyltransferase 1, acyl-CoA wax alcohol acyltransferase 2, and human DGAT candidate gene 3, respectively. Each composition set forth in this Table preferably comprises a preservative, a salt, and a buffering agent. Specific examples of formulations for such compositions are set forth below in this section. In one embodiment, the lipid component of each composition below, and other composition of this invention, liquidefies at a temperature of between 30° C. and 35° C. (e.g. 30° C., 31° C., 32° C., 33° C., 34° C. or 35° C.)

TABLE 3
Examples of Lipid Combinations

| COMPOSITION | DGAT2 | MGAT1 | MGAT2 | MGAT3 | AWAT1 | AWAT2 | hDC3 |
|---|---|---|---|---|---|---|---|
| 1 | X | | | | | | |
| 2 | | X | | | | | |
| 3 | | | X | | | | |
| 4 | | | | X | | | |
| 5 | | | | | X | | |
| 6 | | | | | | X | |
| 7 | | | | | | | X |
| 8 | X | X | | | | | |
| 9 | X | | X | | | | |
| 10 | X | | | X | | | |
| 11 | X | | | | X | | |
| 12 | X | | | | | X | |
| 13 | X | | | | | | X |
| 14 | | X | X | | | | |
| 15 | | X | | X | | | |
| 16 | | X | | | X | | |
| 17 | | X | | | | X | |
| 18 | | X | | | | | X |
| 19 | | | X | X | | | |
| 20 | | | X | | X | | |
| 21 | | | X | | | X | |
| 22 | | | X | | | | X |
| 23 | | | | X | X | | |
| 24 | | | | X | | X | |
| 25 | | | | X | | | X |
| 26 | | | | | X | X | |
| 27 | | | | | X | | X |
| 28 | | | | | | X | X |
| 29 | X | X | X | | | | |
| 30 | X | X | | X | | | |
| 31 | X | X | | | X | | |
| 32 | X | X | | | | X | |
| 33 | X | X | | | | | X |
| 34 | X | | X | X | | | |
| 35 | X | | X | | X | | |
| 36 | X | | X | | | X | |
| 37 | X | | X | | | | X |
| 38 | X | | | X | X | | |
| 39 | X | | | X | | X | |
| 40 | X | | | X | | | X |
| 41 | X | | | | X | X | |
| 42 | X | | | | X | | X |
| 43 | X | | | | | X | X |
| 44 | | X | X | X | | | |
| 45 | | X | X | | X | | |
| 46 | | X | X | | | X | |
| 47 | | X | X | | | | X |
| 48 | | X | | X | X | | |
| 49 | | X | | X | | X | |
| 50 | | X | | X | | | X |
| 51 | | X | | | X | X | |
| 52 | | X | | | X | | X |
| 53 | | X | | | | X | X |
| 54 | | | X | X | X | | |
| 55 | | | X | X | | X | |
| 56 | | | X | X | | | X |
| 57 | | | X | | X | X | |
| 58 | | | X | | X | | X |
| 59 | | | X | | | X | X |
| 60 | | | | X | X | X | |
| 61 | | | | X | X | | X |
| 62 | | | | X | | X | X |
| 63 | | | | | X | X | X |
| 64 | X | X | X | X | | | |
| 65 | X | X | X | | X | | |
| 66 | X | X | X | | | X | |
| 67 | X | X | X | | | | X |
| 68 | X | X | | X | X | | |
| 69 | X | X | | X | | X | |
| 70 | X | X | | X | | | X |
| 71 | X | X | | | X | X | |
| 72 | X | X | | | X | | X |
| 73 | X | X | | | | X | X |
| 74 | X | | X | X | X | | |
| 75 | X | | X | X | | X | |
| 76 | X | | X | X | | | X |
| 77 | X | | X | | X | X | |
| 78 | X | | X | | X | | X |
| 79 | X | | X | | | X | X |
| 80 | X | | | X | X | X | |
| 81 | X | | | X | X | | X |
| 82 | X | | | X | | X | X |
| 83 | X | | | | X | X | X |
| 84 | | X | X | X | X | | |
| 85 | | X | X | X | | X | |
| 86 | | X | X | X | | | X |
| 87 | | | X | X | X | X | |
| 88 | | | X | | X | X | X |
| 89 | | | | X | X | X | X |
| 90 | X | X | X | X | X | | |
| 91 | X | X | X | X | | X | |
| 92 | X | X | X | X | | | X |
| 93 | X | X | X | | X | X | |
| 94 | X | X | X | | X | | X |
| 95 | X | X | X | | | X | X |
| 96 | X | X | | X | X | X | |
| 97 | X | X | | X | X | | X |
| 98 | X | X | | X | | X | X |
| 99 | X | X | | | X | X | X |
| 100 | X | | X | X | X | X | |
| 101 | X | | X | X | X | | X |
| 102 | X | | X | X | | X | X |
| 103 | X | | X | | X | X | X |
| 104 | X | | | X | X | X | X |
| 105 | | X | X | X | X | X | |
| 106 | | X | X | X | X | | X |
| 107 | | X | X | X | | X | X |
| 108 | | X | X | | X | X | X |
| 109 | | X | | X | X | X | X |
| 110 | | | X | X | X | X | X |
| 111 | X | X | X | X | X | X | |
| 112 | X | X | X | X | X | | X |
| 113 | X | X | X | X | | X | X |
| 114 | X | X | X | | X | X | X |
| 115 | X | X | | X | X | X | X |
| 116 | X | | X | X | X | X | X |
| 117 | | X | X | X | X | X | X |
| 118 | X | X | X | X | X | X | X |

### Example 3

### Formulations

The aqueous compositions of the invention can be administered, for example, in the following formulations:

Formulation A

| | |
|---|---|
| 0.055% | monobasic sodium phosphate |
| 0.227% | anhydrous dibasic sodium phosphate |
| 0.6% | sodium chloride |
| 0.75% | potassium chloride |
| 0.003% | anhydrous dextrose |
| 0.1% | Dextran 70 |
| 0.8% | hydroxypropyl methyl chloride |
| 0.01% | benzalkonium chloride |
| 1.0% | lipid component |
| 96.455% | purified or sterile water |

(U.S. Pat. No. 5,681,148, issued Jan. 19, 1990 (Smith))

Formulation B

| | |
|---|---|
| 0.055% | monobasic sodium phosphate |
| 0.227% | anhydrous dibasic sodium phosphate |
| 0.6% | sodium chloride |
| 0.75% | potassium chloride |
| 0.003% | anhydrous dextrose |
| 0.1% | Dextran 70 |
| 0.8% | hydroxypropyl methyl chloride |
| 0.01% | benzalkonium chloride |
| 4.0% | lipid component |
| 93.455% | purified or sterile water |

(U.S. Pat. No. 5,681,148, issued Jan. 19, 1990 (Smith))

Formulation C

| | |
|---|---|
| 0.055% | monobasic sodium phosphate |
| 0.227% | anhydrous dibasic sodium phosphate |
| 0.6% | sodium chloride |
| 0.75% | potassium chloride |
| 0.003% | anhydrous dextrose |
| 0.1% | Dextran 70 |
| 0.8% | hydroxypropyl methyl chloride |
| 0.01% | benzalkonium chloride |
| 0.10% | lipid component |
| 97.355% | purified or sterile water |

(U.S. Pat. No. 5,681,148, issued Jan. 19, 1990 (Smith))

Formulation D

| | |
|---|---|
| 0.001% | Lipid Component |
| 15.0% | Polyethylene Glycol 300 |
| 5.0% | Polyoxyl 40 Stearate |
| 0.5% | Chlorobutanol |
| 3.0% | Povidone |
| q.s. 100 | Purified water |

(U.S. Pat. No. 4,826,871, issued May 2, 1989 (Gressel et al.))

### Example 4

### Preparation of Formulations

A suspension of the composition of the invention is prepared as follows:

Nine grams of lipid is added slowly while stirring to 40 grams of buffer solution including 1 liter of water, 7.1 grams of disodium hydrogen phosphate, and 6.9 grams of sodium dihydrogen phosphate monohydrate at 90° C. The pH is 6.8. 40 ml of phosphate buffer and 0.15 g cholorbutanol are added.

Alternatively, the lipid can be added to the solution after it has cooled to room temperature and is reheated to 90° C.

The mixture is stirred for 4 minutes until the temperature falls to 40° C. It is then poured onto a pan of polyvinylchloride to a thickness which will give a dry thickness in the range from approximately 0.4 mm to 0.7 mm. The resulting film is dried at room temperature for one day.

A solution of formaldehyde (1% by weight) is prepared by addition of 13.1 grams of 38% formaldehyde reagent to 487 grams phosphate buffer (pH 6.8). The lipid composition films are submerged in this buffered formaldehyde solution for 20 minutes at room temperature. They are quickly rinsed with water and soaked in ice water for 2 hours. The films are removed from the ice water and dried overnight.

The dried film is then cut into individual particles using a mold cutter. The mold cutter forms disc-shaped particles having a diameter of 1 mm, and a thickness of 0.4 mm, which was the thickness of the dried film.

A liquid carrier medium made of sterile distilled water, 1% w. poly vinyl alcohol and 0.004% benzalkonium chloride is prepared. A suspension of about 5 lipid composition particles per 0.25 cc of carrier medium is prepared. About 5 globules, with dimensions of about 0.5 mm by 1 mm by 1 mm, of semi-solid petrolatum are added. The liquid suspension can be administered from a dispenser by dropping the suspension into the eye.

(U.S. Pat. No. 4,923,700, issued May 8, 1990 (Kaufman))

### REFERENCES FOR EXAMPLES 1-4

U.S. Pat. No. 4,826,871, issued May 2, 1989 (Gressel et al.).
U.S. Pat. No. 4,923,700, issued May 8, 1990 (Kaufman).
U.S. Pat. No. 5,681,148, issued Jan. 19, 1990 (Smith).
Buhman, K. F., Accad, M., and Farese, R. V. (2000).
Mammalian acyl-CoA: cholesterol acyltransferases. Biochim Biophys Acta 1529, 142-154.
Buhman, K. K., Chen, H. C., and Farese, R. V., Jr. (2001).
The Enzymes of Neutral Lipid Synthesis. J Biol Chem 276(44):40369-72.
Farese, R. V., Jr. (1998). Acyl CoA:cholesterol acyltransferase genes and knockout mice. Curr Opin Lipidol 9, 119-123.
Oelkers, P., Behari, A., Cromley, D., Billheimer, J. T., and Sturley, S. L. (1998), Characterization of two human genes encoding acyl coenzyme A:Cholesterol acyltransferase-related enzymes. J Biol Chem 273, 26765-26771.
Oelkers, P. M., and Sturley, S. L. (2004). Mechanisms and mediators of neutral lipid biosynthesis in eukaryotic cells. In Lipid metabolism and membrane biogenesis, G. Daum, ed. (Berlin, Springer-Verlag), pp. 281-311.
Sturley, S. L. (1997). Molecular Aspects of Intracellular Sterol Esterification The Acyl Coenzyme A:Cholesterol Acyltransferase (ACAT) reaction. Curr Opin Lipidol 8, 167-173.
Turkish, A. R., Henneberry, A. L., Cromley, D., Padamsee, M., Oelkers, P., Bazzi, H., Christianno, A. M., Billheimer,
J. T., and Sturley, S. L. (2004). Identification of Two Novel Human Acyl-CoA Wax Alcohol Acyltransferases: Members of the Diacylglycerol Acyltransferase 2 (DGAT2) Gene Superfamily. J Biol Chem 280, 14755-14764.
Yang, H., Cromley, D., Wang, H., Billheimer, J. T., and Sturley, S. L. (1997). Functional Expression of a cDNA to Human acyl-CoA:cholesterol acyltransferase (ACAT) in Yeast; Species-Dependent Substrate Specificity and Inhibitor Sensitivity. J Biol Chem 272, 3980-3985.

### Example 5

### Eukaryotic Neutral Lipid Synthesis

In mammals, the cytoplasmic storage of fatty acids, sterols, and alcohols as neutral lipids provides reservoirs for steroidogenesis, bile acid synthesis, lipoprotein trafficking, membrane formation/maintenance, and epidermal integrity. Cholesteryl ester (CE), triglyceride (TG), and wax ester (WE) synthesis provides a method for detoxification of fatty acids and alcohols. In addition, triacylglycerol, a fatty acyl ester derivative of glycerol, is the major energy depot of all eukaryotic and some bacterial cells. The energy of oxidation of the alkyl chains of TG (38 KJ/g) is more than twice the same weight of carbohydrate or protein, and unlike polysaccharide, TG carries no extra weight as water of solvation. Moreover, TG is segregated into cytoplasmic lipid droplets and thus has no effect on the osmolarity of the cytosol.

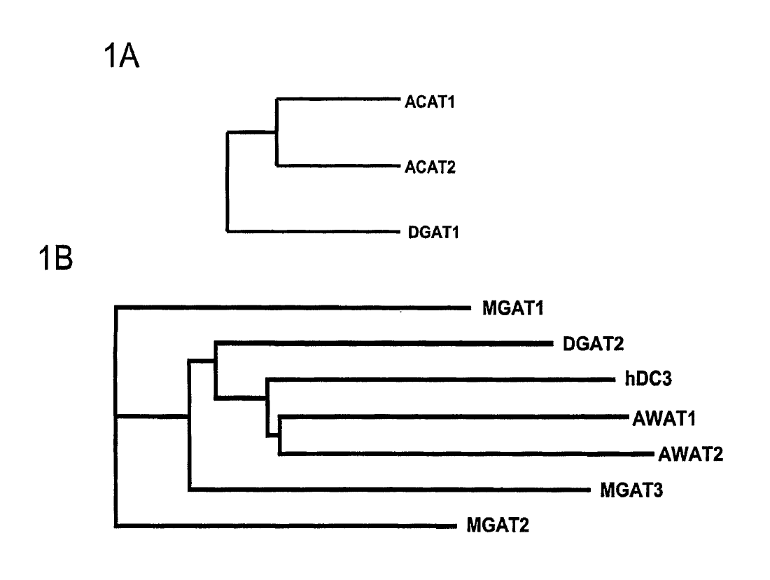

Given such critical roles in cellular function, it is not surprising that many distinct pathways and genes exist for the production of neutral lipids (FIG. 1), and that they are conserved across kingdoms (17). In this review, we will focus on this enzyme redundancy and the concept that this may permit manipulation of the individual reactions as therapeutic targets in several disease pathologies.

Molecular Aspects of Sterol Esterification

In mammals, sterols are esterified with fatty acids but are biologically active (and potentially cytotoxic) in the non-acylated free form, where they are contribute to membrane stability and act as substrates for steroid hormones or bile acids. In all eukaryotic cells, steryl esters are stored in cytoplasmic lipid droplets. In mammalian liver or intestine, CE is packaged into lipoprotein particles in the endoplasmic reticulum for redistribution via the circulation. The esterification reaction thus represents a critical, evolutionary conserved step in sterol homeostasis. However, the accretion of CE in macrophages or smooth muscle cells leads to the formation of foam cells along the arterial wall, and has been implicated in the development of atherosclerotic lesions. This has sparked interest in the elucidation of the mediators and mechanisms of these reactions, with hopes of treating atherosclerosis and/or hypercholesterolemia.

Two Acyl-CoA cholesterol acyltransferase (ACAT) genes (1 and 2) have been identified and characterized in mammals (reviewed; (4, 9, 17)). ACAT1 is widely expressed with the highest levels in macrophages, steroidogenic tissues, and sebaceous glands as well as atherosclerotic lesions. ACAT2, on the other hand, is limited in its expression to the liver and small intestine (4, 17). These findings suggest that ACAT1 primarily incorporates cholesterol esters into cytoplasmic lipid droplets, while ACAT2 is involved with lipoprotein assembly in the enterohepatic system. However, it remains unclear as to which ACAT predominantly operates in the human liver. Immunodepletion experiments (7) indicate that ACAT1 accounts for more than 90% of ACAT activity in adult liver autopsy tissue although it is the less abundant transcript. By contrast in the intestine, the majority of ACAT activity is determined by ACAT2.

The ACATs are regulated by translational and post-translational mechanisms (reviewed; (4)). ACAT1 mRNA is increased in the liver and aorta following a cholesterol rich diet and after exposure to free fatty acids in human HepG2 cells. For the most part, however, ACAT1 is allosterically activated by cholesterol and oxysterols but not fatty acids. It seems likely given the degree of sequence conservation, that ACAT2 is also allosterically regulated by cholesterol although transcriptional regulation of this isoform is observed.

Conservation of the ACAT Reaction and Consequences of the Loss of Sterol Esterification

Esterification of sterols has been conserved throughout eukaryotic evolution. In yeast, ACAT related enzymes 1 & 2 (ARE1 & ARE2) are the orthologs of mammalian ACATs. Although deletion of ARE1 and ARE2 eliminates sterol esterification in yeast, the cells are viable due to downregulation of ergosterol synthesis. Deletion of ARE2 leads to a marked reduction in ergosterol esterification, reflecting its substrate preference for the major free sterol in yeast. ARE1 esterifies a wider range of sterols and thus likely serves to detoxify other sterols within the cell (17).

Further insight into the role of each of the ACATs has been gleaned from knockout models in mice. Mice with partial ACAT1 activity (Acact1−/−) are healthy with normal serum cholesterol, intestinal cholesterol absorption and hepatic ACAT activity (owing to the presence of ACAT2). However, ACAT1 null mice (Acat1−/−) suffer from dry eyes due to atrophic meibomian glands and have marked depletion of cholesterol esters in the adrenal glands and cultured peritoneal macrophages (1). However, ACAT1 deficiency may promote, not prevent plaque formation in atherosclerosis susceptible models. In contrast, mice lacking ACAT2 (Acat2−/−) have impaired dietary cholesterol absorption, are resistant to diet induced hypercholesterolemia and gallstone formation and are nearly completely protected from atherosclerosis in an ApoEV−/− background (3, 24).

ACAT Inhibitors

ACAT inhibitors have long been sought as potential therapeutics for atherosclerosis. Several animal studies involving nonselective ACAT inhibitors (e.g. avasimibe, pactimibe) showed a promising reduction of atherosclerotic plaques (14). Unfortunately, human trials have proven to be disappointing, likely because generalized ACAT inhibition leads to an increase in free cholesterol levels, apoptosis, and destabilization of ATP binding cassette protein Al (ABCA1), a crucial mediator of reverse cholesterol transport (14). However, ACAT2 specific inhibition may be a viable option for future pharmacologic intervention. Atherosclerotic susceptible mice that were given liver specific antisense oligonucleotides targeting ACAT2 were resistant to diet-induced hypercholesterolemia and had less aortic atherosclerotic lesions (2).

Molecular Aspects of Diacylglycerol Esterification

Diacylglycerol (DAG), the obligate precursor to TG, is derived either from the glycerol-3-phosphate pathway or the monoacylglycerol pathway whereby 2-monoacylglycerol, a lipolysis product of triglyceride, is re-esterified. Whatever its source in mammals, diacylglycerol is esterified to triglyceride by an acyl CoA: diacylglycerol acyltransferase (DGAT) reaction (17). There are at least two independent mammalian enzymes known to catalyze this reaction, DGAT1 and DGAT2 (11). DGAT1 is closely related to the ACATs (FIG. 1A); the divergence in its amino acid sequence re-defines its substrate specificity to diacylglycerol (5). DGAT2 defines an unrelated gene family (FIG. 1B). Both enzyme families are conserved across multiple organisms including yeast and plants (17).

In humans, DGAT1 is highly expressed in human small intestine, colon, testis and skeletal muscle, but has notably lower levels of expression in adipose and liver (5, 16). Mice lacking DGAT1 (Dgat1−/−) were found to have normal plasma TG levels, suggesting other mechanisms by which mammals synthesize TG (19). Subsequently, DGAT2, the original member of the second human DGAT family, was identified by sequence similarity to proteins purified from Mortierella ramanniana, an oleaginous fungus (6). Members of the DGAT2 family have no sequence homology to the ACAT family, including DGAT1. They are likely derived from an ancestor with lysophosphatidic acid acyltransferase (LPAAT) activity, required for the final steps in phosphatidic acid biosynthesis. This homology is maintained in the whole human DGAT2 gene family and includes the conservation of residues at the active sites of bacterial glycerol-3-phosphate acyltransferases.

When expressed in insect or yeast cells, DGAT2 produces robust DGAT activity, and like DGAT1, shows little preference in terms of the fatty acyl-CoA substrate (6). DGAT2 possesses widespread expression in humans, with particularly high levels in liver and adipose tissue (6). The expression patterns of the DGATs indicate that they may have different functions within different tissues. DGAT1 likely plays a role in intestinal repackaging of free fatty acids using the monoacylglycerol pathway, whereas DGAT2 may function primarily in TG synthesis and export from the liver and deposition in adipose tissue.

Triglyceride Biosynthesis is Conserved in Yeast Studies of neutral lipid biosynthesis in yeasts such as Saccharomyces cerevisiae have led to further major insights into the mechanisms and mediators of triglyceride biosynthesis in eukaryotes. In yeast, three structurally different enzymes mediate diacylglycerol esterification (reviewed (17)). By a novel (PDAT; phospholipid diacylglycerol acyltransferase) reaction, the LRO1 gene product, an ortholog of mammalian lecithin-cholesterol acyltransferase (LCAT), mediates TG synthesis up to 75% of the normal strain, depending on culture conditions. LRO1 utilizes phospholipids in an acyl-CoA independent esterification of diacylglycerol, a reaction that predominates during the growth phase of yeast. PDAT (i.e. LRO1) orthologs have been identified in multiple plant species. However, mammalian LCAT specifically uses cholesterol as its substrate. Interestingly, under appropriate conditions, mammalian LCAT is able to esterify diacylglycerol to TG. Presumably as serum lipoprotein trafficking systems evolved, the substrate specificity of the LCAT orthologs shifted from diacylglycerol to sterols and from a subcellular to extracellular location.

The product of DGA1, the sole yeast ortholog of human DGAT2, mediates the majority of TAG synthesis in LRO1 knockout strains (17). A yeast ortholog of ACAT and DGAT1, ARE2, plays a minor role in TAG synthesis (17). Deletion of DGA1, LRO1, ARE1 and ARE2, completely abolishes the ability of yeast to synthesize any TG or steryl ester, thus defining the enzymes responsible for neutral lipid synthesis in yeast. Surprisingly, under standard conditions, the quadruple deletion strains (are1Δare2Δdga1Δiro1Δ) are healthy and have no growth defects (17). It appears from these studies that under stress free conditions, neutral lipid storage is not necessary for survival. Neutral lipids probably play more of a role during periods of nutrient deprivation or cellular stress. In view of this, utilization of yeast strains devoid of background neutral lipid synthetic abilities has facilitated the study of mammalian mediators of neutral lipid metabolism (23).

### Consequences of the Loss of Diacylglycerol Esterification

Aberrant DGAT expression in any of several tissues or organ systems may play a role in disorders such as obesity and non-alcoholic fatty liver disease. DGAT1 mRNA levels increase seven fold when 3T3L1 cells are induced with insulin and dexamethasone to differentiate into adipocytes, leading to a ninety-fold increase in DGAT1 protein and DGAT activity (26). However, when DGAT1 is overexpressed in undifferentiated 3T3L1 cells, a 20 to 40-fold increase in mRNA is associated with a modest 2-3-fold increase in DGAT activity. TG turnover remained stable, and thus, cellular TG mass was doubled, suggesting that DGAT1 is rate limiting in TG synthesis. However, mice that lack DGAT1 (Dgat1−/−) have normal serum triglyceride levels, indicating that DGAT1 does not play an essential role in the export of triglyceride from the liver (19). DGAT2 expression increases 30-fold upon differentiation of 3T3L1 cells and yet further when treated with glucose and insulin (13). Glucose preferentially enhances DGAT1 mRNA expression, whereas insulin increases the level of DGAT2 mRNA. However, when fasted mice are fed a high carbohydrate meal, DGAT2, but not DGAT1 mRNA is increased in liver, adipose and small intestine.

Mice lacking DGAT1 (Dgat 1−/−) exhibit 50% less body fat but are otherwise healthy and fertile with normal serum TG (19). Their observable phenotypes include poor milk production due to deficient TG production in mammary glands and dry fur and hair associated with atrophy of sebaceous glands (19). Dgat1−/− mice are resistant to diet-induced obesity and hepatic steatosis when placed on a high fat diet. Although intestinal DGAT activity is reduced, there are no differences in fatty acid absorption in Dgat1−/− mice. Rather, the decreased body fat and lack of weight gain in these mice is due to an increase in total energy expenditure due to physical activity and thermogenesis (8, 19).

Dgat1−/− mice also exhibit significant metabolic changes that represent a physiologically sound alteration in health. Mice lacking DGAT1 have lower plasma glucose levels associated with increased insulin and leptin sensitivity. The importance of leptin on DGAT1 physiology and its role in obesity was demonstrated when DGAT1 deficiency was introduced into obese leptin resistant Agouti yellow, and leptin knockout ob/ob mice. Although leptin resistant mice were protected against obesity and insulin resistance when they lacked DGAT1, the same could not be said regarding mice that lack leptin, demonstrating that the effects of DGAT1 deficiency are dependent on intact leptin function (8). Thus, the precise role of leptin in DGAT physiology remains unknown.

Furthermore, transplantation of white adipose tissue from DGAT1 deficient mice into wild type mice placed on a high fat diet confers obesity resistance and improved insulin sensitivity (8). This suggests that factors secreted from adipocytes in a DGAT1 deficiency state are responsible for the improved body fat, glucose disposal, and energy expenditure. Adiponectin, an adipocyte secreted factor that stimulates energy expenditure was ruled out in that mice lacking both adiponectin and DGAT1 continue to be protected from obesity and hepatic steatosis (22).

These approaches were complemented by studies in which DGAT1 was overexpressed in cell culture or in specific tissues (15). Transgenic mice that overexpress DGAT1 in white adipose tissue become obese due to adipocyte TG deposition, but surprisingly are insulin sensitise and have normal glucose disposal. Overexpression of DGAT1 in adipocytes of obese-resistant FVB mice, prompted hepatic steatosis in association with obesity-resistance, elevated plasma free fatty acids, and insulin and leptin resistance.

Little is known about DGAT expression and regulation within the liver. Two topological types of DGAT activities have been described biochemically in studies using rat liver microsomes: an overt DGAT activity associated with cytosolic droplet TAG synthesis, and a latent DGAT activity in the lumen of the endoplasmic reticulum that may be responsible for TAG secretion in lipoprotein particles (18). However, the expression of DGAT1 and DGAT2 does not correlate with either of these DGAT activities. Overexpression of human DGAT1 in rat hepatoma McA-RH7777 cells increases synthesis, cellular accumulation, and secretion of TG (12). This is associated with decreased intracellular degradation of newly synthesized apolipoprotein B. In support of these findings, mice overexpressing DGAT1 via an adenoviral-mediated gene transfection exhibit increased hepatic VLDL secretion. The precise role of DGAT1 in the development of fatty liver syndromes such as non-alcoholic fatty liver disease (NAFID) remains to be determined. However in a small study, DGAT1 expression was found to be upregulated in the livers of individuals with this syndrome (15).

Mice deficient in DGAT2 (Dgat2−/−) are severely depleted of triglycerides in their tissues and plasma, leading to neonatal death from metabolic disarray and poor skin barrier function (21). DGAT1 was unable to compensate for the loss of DGAT2, suggesting different roles for the two enzymes, and that DGAT2 is the enzyme responsible for the majority of TAG synthesis in mice and may not be an ideal target for treatment of obesity disorders. Conversely, a marked reduction in hepatic triglyceride content and steatosis arose when hepatic DGAT2 expression was reduced with antisense oligonucleotides in wild type and ob/ob obese mice on high fat diets (25). Several studies found that when hepatic DGAT2 is overexpressed in mice, liver TG mass increases without a concomitant change in VLDL secretion. Thus, DGAT2 may be a legitimate target for dyslipidemic specific disorders such as NAFLD.

The DGAT2 Gene Family

As described here, eukaryotic cells have evolved at least three independent mechanisms to neutralize/store alcohols, such as sterols and diacylglycerols. In humans, the DGAT2 gene family is even more complex, comprising six additional members, all of which synthesize TG in vitro. Three members of this family are autosomally encoded acyl-CoA:monoacylglycerol acyltransferases (MGATs 1-3) that direct the synthesis of diacylglycerol by esterification of monoacylglycerol. Two encode acyl-CoA wax alcohol acyltransferases (AWATs 1-2) that synthesize waz esters by esterification of long chain alcohols. The latter reaction appears to be particularly important in the sebocyte, where wax esters comprise a significant component of sebaceous gland secretions. The final member of this gene family, hDC3 (initially termed DC3 by Farese), has recently been characterized with regard to substrate preference and reaction product. Unique for this group of genes, hDC3 catalyzes the synthesis of a di-esterified form of ling chain dialcohols. It also has robust triglyceride synthetic activity. This gene has been renamed ADAT1 (acylCoA dialcohol acyltransferase 1) based on its major activity. Diesterified long chain alcohols (wax diesters) are a major component of the tear gland secretion (meibum) and as such have great potential as lipid replacements in the artificial tear formulations described herein.

The MGAT reaction provides an alternative to the Kennedy pathway for the synthesis of diacylglycerol that is particularly important for dietary fat absorption at the intestinal enterocyte, where TG is lipolyzed to monoacylglycerol and free fatty acids, and then resynthesized and secreted in chylomicrons. However, it is unknown why there 3 MGATs in humans. It has been postulated that the existence of multiple MGATs may be related to specific tissue expression patterns. For example, mouse MGAT1 is expressed in most tissues but not in the intestine. MGATs 2 and 3 are primarily expressed in the intestine and likely are the key contributors to TG repackaging within the enterocyte. Interestingly, MGAT3 transcripts are most abundant in the ileum, distal to the regions of maximum lipid absorption. The MGATs are also partially defined by their substrate specificity in that MGAT3 prefers 2-monoacylglycerol, the predominant product of TG lipolysis in the intestinal tract, but MGATs 1 & 2 do not have such substrate specificity. The MGATs also exhibit some DGAT activity and so the precise physiologic roles of these members of the DGAT2 gene family remain to be determined.

Interestingly, the remaining members of the human DGAT2 gene family belong to an X-linked cluster (Xq13.1) of approximately 200 Kilobase pairs (FIG. 1B). All three enzymes are highly expressed in the skin and possess significant DGAT activity; however, two of these (AWAT 1 & 2) were found to predominantly mediate the esterification of various fatty alcohols and fatty acids into wax esters via an acyl-CoA wax alcohol acyltransferase reaction (23). A murine wax synthase ortholog of AWAT2 with similar fatty acyl-CoA and fatty alcohol preferences was also reported (10). Wax esters are abundant in the cuticle of plants, insect exoskeleton coating, and mammalian sebum where they likely act as permeability barriers.

The existence of two AWATs can be explained by their different expression and substrate specificity patterns. Within the sebaceous gland, the peripheral layer of sebocytes is comprised of undifferentiated cells deficient in lipid droplets. As cells shift toward the center of the gland, they mature and accumulate lipid droplets (20). A WAT expression is limited to the sebaceous gland in a differentiation specific manner (23). AWAT2 is restricted to undifferentiated peripheral sebocytes, while AWAT1 is expressed in more mature, centrally located cells. The expression and substrate specificity pattern of the AWATs suggests that wax ester metabolism reflects and may play an important role in sebocyte differentiation. It is possible that as immature sebocytes differentiate, the wax esters undergo hydrolysis and reesterification in order to optimize the fatty acid and alcohol saturation and chain length within the sebaceous mileu, thus providing maximum hydrophobicity and protection for the skin. One may postulate, then, that aberrant expression of the AWATs may lead to any number of disorders in which the lipid composition' of the skin is awry, such as acne vulgaris or ocular rosacea.

There are multiple pathways to the formation of neutral lipids in eukaryotes. These reactions provide a critical resource for many distinct cellular processes and their loss is often catastrophic but not immediately fatal. The importance of these reactions is further demonstrated by the fact that they are conserved across many billions of years of evolution and have arisen independently at least 3 times. This apparent redundancy in neutral lipid synthesis is obviously advantageous; marked changes in lipid homeostasis arise when expression of the DGATs is altered in mammals, but embryonic lethality is not a consequence. Further clarification of the metabolic pathways of neutral lipid synthesis may hasten the development of therapeutic interventions for several diseases. It is interesting to speculate that isoform-specific inhibition of DGAT1, DGAT2, ACAT2 or the AWATs, may be effective and non-toxic therapeutics for type 2 diabetes and obesity, NAFLD, hyperlipidemia and atherosclerosis, or acne, respectively.

### REFERENCES FOR EXAMPLE 5

1. Accad M, Smith S J, Newland D L, Sanan D A, King L E, Jr., Linton M F, Fazio S, and Farese R V, Jr. Massive xanthomatosis and altered composition of atherosclerotic lesions in hyperlipidemic mice lacking acyl CoA: cholesterol acyltransferase 1. J Clin Invest 105: 711-719., 2000.
2. Bell T A, 3rd, Brown J M, Graham M J, Lemonidis K M, Crooke R M, and Rudel L L. Liver-specific inhibition of acyl-coenzyme axholesterol acyltransferase 2 with antisense oligonucleotides limits atherosclerosis development in apolipoprotein BlOO-orily low-density lipoprotein receptor−/− mice. Arterioscler Thromb Vase Biol 26: 1814-1820, 2006.
3. Buhman K K, Accad M, Novak S, Choi R S, Wong J S, Hamilton R L, Turley S, and Farese R V, Jr. Resistance to diet-induced hypercholesterolemia and gallstone formation in ACAT2-deficient mice. Nat Med 6: 1341-1347, 2000.
4. Buhman K K, Chen H C, and Farese R V, Jr. The Enzymes of Neutral Lipid Synthesis. J Biol Chem, 2001.
5. Cases S, Smith S J, Zheng Y W, Myers H M, Lear S R, Sande E, Novak S, Collins C, Welch C B, Lusis A J, Erickson S K, and Farese R V, Jr. Identification of a gene encoding an acyl CoA:diacylglycerol acyltransferase, a key enzyme in triacylglycerol synthesis. Proc Natl Acad Sci USA 95: 13018-13023, 1998.
6. Cases S, Stone S J, Zhou P, Yen E, Tow B, Lardizabal K D, Voelker T, and Farese R V, Jr. Cloning of DGAT2, a second mammalian diacylglycerol acyltransferase, and related family members. J Biol Chem 276: 38870-38876., 2001.
7. Chang C C, Sakashita N, Ornvold K, Lee O, Chang E T, Dong R, Lin S, Lee C Y, Strom S C, Kashyap R, Fung J J, Farese R V, Jr., Patoiseau J F, Delhon A, and Chang T Y. Immunological quantitation and localization of ACAT-1 and ACAT-2 in human liver and small intestine. J Biol Chem 275: 28083-28092, 2000.
8. Chen H C, Jensen D R, Myers H M, Eckel R H, and Farese R V, Jr. Obesity resistance and enhanced glucose metabolism in mice transplanted with white adipose tissue lacking acyl CoArdiacylglycerol acyltransferase 1. J Clin Invest 111: 1715-1722, 2003.
9. Cheng D, Liu J, C. C. Y. C, and Chang T Y. Mammalian ACAT and DGAT2 gene families. In: Lipid metabolism and membrane biogenesis, edited by Daum G. Berlin: Springer-Verlag, 2004, p. 241-265.
10. Cheng J B and Russell D W. Mammalian wax biosynthesis II: Expression cloning of wax synthase cDNAs encoding a member of the acyltransferase enzyme family. J Biol Chem, 2004.
11. Farese R V, Jr., Cases S, and Smith S J. Triglyceride synthesis: insights from the cloning of diacylglycerol acyltransferase. Curr Opin Lipidol 11: 229-234, 2000.
12. Liang J J, Oelkers P, Guo C, Chu P C, Dixon J L, Ginsberg H N, and Sturley S L. Overexpression of human diacylglycerol acyltransferase 1, acyl-CoAxholesterol acyltransferase 1 or acyl-CoAxholesterol acyltransferase 2 stimulates secretion of apolipoprotein B-containing lipoproteins in McA-RH7777 cells. J Biol Chem, 2004.
13. Meegalla R L, Billheimer J T, and Cheng D. Concerted elevation of acyl-coenzyme A.diacylglycerol acyltransferase (DGAT) activity through independent stimulation of mRNA expression of DGAT 1 and DGAT2 by carbohydrate and insulin. Biochem Biophys Res Cammun 298: 317-323, 2002.
14. Meuwese M C, Franssen R, Stroes E S, and Kastelein J J. And then there were acyl coenzyme Axholesterol acyl transferase inhibitors. Curr Opin Lipidol 17: 426-430, 2006.
15. Millar J S, Stone S J, Tietge U J, Tow B, Billheimer J T, Wong J S, Hamilton R L, Farese R V, Jr., and Rader D J. Short-term overexpression of DGAT 1 or DGAT2 increases hepatic triglyceride but not VLDL triglyceride or apoB production. J Lipid Res 47: 2297-2305, 2006.
16. Oelkers P, Behari A, Cromley D, Billheimer J T, and Sturley S L. Characterization of two human genes encoding acyl coenzyme A: Cholesterol acyltransferase-related enzymes. J Biol Chem 273: 26765-26771, 1998.
17. Oelkers P M and Sturley S L. Mechanisms and mediators of neutral lipid biosynthesis in eukaryotic cells. In: Lipid metabolism and membrane biogenesis, edited by Daum G. Berlin: Springer-Verlag, 2004, p. 281-311.
18. Owen M R, Corstorphine C C, and Zammit V A. Overt and latent activities of diacylglycerol acytransferase in rat liver microsomes: possible roles in very-low-density lipoprotein triacylglycerol secretion. Biochem J 323: 17-21, 1997.
19. Smith S J, Cases S, Jensen D R, Chen H C, Sande E, Tow B, Sanan D A, Raber J, Eckel R H, and Farese R V, Jr. Obesity resistance and multiple mechanisms of triglyceride synthesis in mice lacking Dgat. Nat. Genet 25: 87-90, 2000.
20. Stewart M E and Downing D T. Chemistry and function of mammalian sebaceous lipids. Adv Lipid Res 24: 263-301, 1991.
21. Stone S J, Myers H M, Watkins S M, Brown B E, Feingold K R, Elias P M, and Farese R V, Jr. Lipopenia and Skin Barrier Abnormalities in DGAT2-deficient Mice. J Biol Chem 279: 11767-11776, 2004.
22. Streeper R S, Koliwad S K, Villanueva C J, and Farese R V Jr. Effects of DGAT1 deficiency on energy and glucose metabolism are independent of adiponectin. Am JPhysiol Endocrinol Metab 291: E388-394, 2006.
23. Turkish A R, Henneberry A L, Cromley D, Padamsee M, Oelkers P, Bazzi H, Christiano A M, Billheimer J T, and Sturley S L. Identification of two novel human acyl-CoA wax alcohol acyltransferases: members of the diacylglycerol acyltransferase 2 (DGAT2) gene superfamily. J Biol Chem 280: 14755-14764, 2005.
24. Willner E L, Tow B, Buhman K K, Wilson M, Sanan D A, Rudel L L, and Farese R V, Jr. Deficiency of acyl CoAxholesterol acyltransferase 2 prevents atherosclerosis in apolipoprotein E-deficient mice. Proc Natl Acad Sci USA 100: 1262-1267, 2003.
25. Yu X X, Murray S F, Pandey S K, Booten S L, Bao D, Song X Z, Kelly S, Chen S, McKay R, Monia B P, and Bhanot S. Antisense oligonucleotide reduction of DGAT2 expression improves hepatic steatosis and hyperlipidemia in obese mice. Hepatology 42: 362-371, 2005.
26. Yu Y H, Zhang Y, Oelkers P, Sturley S L, Rader D J, and Ginsberg H N. Posttranscriptional control of the expression and function of diacylglycerol acyltransferase-1 in mouse adipocytes. J Biol Chem 277: 50876-50884, 2002.

1. An aqueous composition suitable for use as an artificial tear solution comprising one or more lipids produced by an enzyme of the diacylglycerol acyltransferase 2 (DGAT2) family and/or the acyl-CoA cholesterol acytransferase (ACAT) family.

2. The aqueous composition of claim 1, wherein the composition comprises lipids produced by two enzymes of the DGAT2 and/or ACAT families.

3. The aqueous composition of claim 1, wherein the composition comprises lipids produced by three enzymes of the DGAT2 and/or ACAT families.

4. The aqueous composition of claim 1, wherein the enzyme is acyl-CoA wax alcohol acyltransferase 1 (AWAT1) or acyl-CoA wax alcohol acyltransferase 2 (AWAT2).

5. The aqueous composition of claim 2, wherein the enzymes are AWAT1 and AWAT2.

6. The aqueous composition of claim 1, further comprising (i) a preservative, (ii) a salt and/or (iii) a buffering agent.

7. The aqueous composition of claim 6, wherein the composition comprises (i) a preservative, (ii) a salt, and (iii) a buffering agent.

8. An aqueous composition suitable for use as an artificial tear solution comprising a lipid selected from the group consisting of a steryl ester, a triacylglycerol, a diacylglycerol, a wax ester and a dialcohol wax ester.

9. The aqueous composition of claim 8, wherein the steryl ester is approximately 77 percent saturated.

10. The aqueous composition of claim 8, wherein the triacylglycerol is approximately 38 percent saturated.

11. The aqueous composition of claim 8, wherein the waxy ester is approximately 23 percent saturated.

12. The aqueous composition of claim 8, further comprising (i) a preservative, (ii) a salt and/or (iii) a buffering agent.

13. The aqueous composition of claim 12, wherein the composition comprises (i) a preservative, (ii) a salt, and (iii) a buffering agent.

14-17. (canceled)

18. A method for lubricating the surface of a subject's eye comprising topically administering an effective amount of the composition of claim 1 to the subject's eye.

19. The method of claim 18, wherein the subject is undergoing, or is about to undergo, an ocular procedure.

20. The method of claim 18, wherein the subject is a human.

21. A method for lubricating the surface of a subject's eye comprising topically administering an effective amount of the composition of claim 8 to the subject's eye.

22. The method of claim 21, wherein the subject is undergoing, or is about to undergo, an ocular procedure.

23. The method of claim 21, wherein the subject is a human.

24. A method for treating a subject afflicted with dry eye syndrome comprising topically administering a therapeutically effective amount of the composition of claim 1 to one or both of the subject's eyes.

25. The method of claim 24, wherein the subject is undergoing, or is about to undergo, an ocular procedure.

26. The method of claim 24, wherein the subject is a human.

27. A method for treating a subject afflicted with dry eye syndrome comprising topically administering a therapeutically effective amount of the composition of claim 8 to one or both of the subject's eyes.

28. The method of claim 27, wherein the subject is undergoing, or is about to undergo, an ocular procedure.

29. The method of claim 27, wherein the subject is a human.

30-39. (canceled)

40. A method for producing the aqueous composition of claim 1 comprising admixing (i) an aqueous solution with (ii) one or more lipids produced by an enzyme of the DGAT2 and/or the ACAT families.

41-42. (canceled)

43. A method for producing the aqueous composition of claim 8 comprising admixing (i) an aqueous solution with (ii) a lipid selected from the group consisting of a triacylglycerol, a diacylglycerol, a waxy ester, a dialcohol wax ester, and a steryl ester.
